(19) United States
(12) Patent Application Publication
(10) Pub. No.: US 2004/0265992 A1
(43) Pub. Date: Dec. 30, 2004

(54) Microchemical chip and method for producing the same

(75) Inventors: Shin Matsuda, Kokubu-shi (JP); Katsuhiko Onitsuka, Kokubu-shi (JP)

Correspondence Address: HOGAN & HARTSON L.L.P., 500 S. GRAND AVENUE, SUITE 1900, LOS ANGELES, CA, 90071-2611, US

(73) Assignee: KYOCERA CORPORATION

(21) Appl. No.: 10/879,888

(22) Filed: Jun. 29, 2004

(30) Foreign Application Priority Data
Jun. 30, 2003 (JP) P2003-187942

(51) Int. Cl. G01R 31/26; C12M 1/34; H01L 21/66

(52) U.S. Cl. 435/287.2; 438/1

(57) ABSTRACT

A microchemical chip includes a base including a channel for causing a fluid to-be-treated to flow therethrough, supply portions connected to the channel, for causing the fluids to-be-treated to flow therefrom into the channel, respectively, and a collection portion from which a fluid in the channel is drawn to the outside. A plurality of fluids to-be-treated are respectively caused to flow from the supply portions into the channel, and the plurality of fluids to-be-treated caused to flow in are merged and subjected to a predetermined treatment, and then the treated fluid is drawn from the collection portion to the outside. In this microchemical chip, the base is formed by attaching a base body made of ceramics and a lid made of glass, and electrodes that are used for capillary migration are formed by being sintered simultaneously with the base body.

### BACKGROUND OF THE INVENTION

1. Field of the Invention

The present invention relates to a microchemical chip in which a predetermined treatment such as a reaction or analysis can be performed with respect to a fluid to-be-treated such as a fluid or a reagent that flows through a small channel, and a method for producing the same. More specifically, the present invention relates to a microchemical chip in which it is possible to mix a plurality of different fluids to-be-treated and then perform a predetermined treatment, for example, as in the case where blood and a reagent are mixed to cause a reaction, and a method for producing the same.

2. Description of the Related Art

In recent years, in the fields of the chemical technology and the biochemical technology, research to perform reaction with a sample or analysis of a sample in a small area has been conducted, and microchemical systems that are miniaturized systems for chemical reactions, biochemical reactions and analysis of samples have been researched and developed, using the Micro Electro Mechanical System (abbreviated as MEMS) technology.

The reaction and the analysis in the microchemical systems are performed with one chip called a microchemical chip in which a microchannel, a micropump, and a microreactor are formed. For example, the following microchemical chip is proposed: a supply port for supplying a fluid such as a sample and a reagent and a collection port for drawing a treated fluid out are formed in a base made of silicon, glass or resin, the supply port and the collection port are connected via a microchannel whose cross-section area is small, and a micropump for sending a fluid to an appropriate position of the channel is provided (see Japanese Unexamined patent Publication JP-A-2002-214241 (pages 4-5, FIG. 1)). Furthermore, a microchemical chip including means for sending a fluid of capillary migration type utilizing the electro-osmosis phenomenon, instead of the micropump, is also proposed (see Japanese Unexamined patent Publication JP-A-2001-108619 (pages 4-5, FIG. 1). In these microchemical chips, the channels are connected at predetermined positions, and fluids are mixed at the junction portion.

In the microchemical system, compared with the conventional systems, the equipment and the techniques are miniaturized, and therefore the surface area of a reaction per unit volume of a sample can be increased so that the reaction time can be reduced significantly. Moreover, it is possible to control the flow rate precisely, so that reaction and analysis can be performed efficiently. Furthermore, the amount of a sample or a reagent necessary for reaction or analysis can be reduced.

The aforementioned Japanese Unexamined patent Publication JP-A-2001-108619 (pages 4-5, FIG. 1) does not describe the material of the base of the microchemical chip of capillary migration type, but a general microchemical chip is formed of a base made of silicon, glass or resin. Therefore, in a conventional microchemical chip of capillary migration type, electrodes used for capillary migration are formed on a base made of silicon, glass or resin by processing for forming a thin film.

However, in a microchemical chip of capillary migration type using a base made of silicon, glass or resin, the adhesiveness between the electrodes used for capillary migration and the base is poor, and therefore a portion where the electrodes and the base are adhered is corroded by a supplied fluid to-be-treated, in particular, chemicals. Therefore, in the microchemical chip of capillary migration type, there is a limitation regarding the fluid to-be-treated that can be supplied, and the use conditions are disadvantageously limited.

### SUMMARY OF THE INVENTION

An object of the invention is to provide a microchemical chip having excellent chemical resistance and wide-range applicability in which there is no limitation regarding a supplied fluid to-be-treated, and to provide a method for producing the same.

The invention provides a microchemical chip comprising:

a base including a channel for causing a fluid to-be-treated to flow therethrough, a plurality of supply portions connected to the channel, for causing a plurality of fluids to-be-treated to flow therefrom into the channel, respectively, and a collection portion which is connected to the channel and from which a fluid in the channel is drawn to the outside;

a supply portion side electrode formed in the supply portion; and

a collection portion side electrode formed in the collection portion,

the base being composed of a base body made of ceramics having a groove portion constituting the channel and a covering member arranged on the base body so as to cover the groove portion,

the supply portion including a supply channel having its one end connected to the channel and its another end connected to a through-hole for supply formed in the covering member,

the collection portion including a though-hole for collection formed in the covering member so as to be connected to a portion on the most downstream side in a flowing direction of the fluid to-be-treated in the channel,

the plurality of fluids to-be-treated being respectively caused to flow from the plurality of supply portions into the channel, the plurality of fluids to-be-treated caused to flow in being merged and subjected to a predetermined treatment, and the treated fluid is drawn from the collection portion to the outside, and

the supply portion side electrode and the collection portion side electrode being sintered simultaneously with the base body, and capillary migration being performed by applying a voltage between the supply portion side electrode and the collection portion side electrode.

According to the invention, the fluids to-be-treated that are supplied from the plurality of supply portions flow through the channel by capillary migration and are drawn from the collection portion to the outside. Therefore, when the plurality of fluids to-be-treated that are different from each other are caused to flow in from the plurality of supply portions, respectively, the plurality of fluids to-be-treated caused to flow in are merged and flow through the channel, and are subjected to a predetermined treatment. Then, the treated fluid is drawn from the collection portion to the outside.

In the invention, the supply portion side electrode and the collection portion side electrode which are used for capillary migration, are sintered simultaneously with the base body made of ceramics, and therefore the adhesiveness between the electrodes and the base body is improved. Thus, a portion where the base body and the electrode are adhered is prevented from being corroded by the fluid to-be-treated, in particular, chemicals, and thus the chemical resistance can be improved. Thus, a microchemical chip having wide applicability in which there is no limitation regarding the supplied fluid to-be-treated can be realized.

In the invention, the supply portion side electrode is formed on a part of a bottom face of the groove portion formed in the base body which part is to be positioned immediately below the through-hole for supply, and

the collection portion side electrode is formed on a part of a bottom face of the groove portion formed in the base body which part is to be positioned immediately below the through-hole for collection.

According to the invention, the electrodes are formed on the bottom face of the groove portion that is a flat surface, so that the adhesiveness between the base body and the electrodes can be further improved. Furthermore, the electrodes can be formed relatively easily.

The invention provides a microchemical chip comprising:

a base made of ceramics and including a channel for causing a fluid to-be-treated to flow therethrough, a plurality of supply portions connected to the channel, for causing a plurality of fluids to-be-treated to flow therefrom into the channel, respectively, and a collection portion which is connected to the channel and from which a fluid in the channel is drawn to the outside;

a supply portion side electrode formed in the supply portion; and

a collection portion side electrode formed in the collection portion,

the base being composed of a base body made of ceramics having a groove portion constituting the channel and a covering member made of ceramics and arranged on the base body so as to cover the groove portion,

the supply portion including a supply channel having its one end connected to the channel and its another end connected to a through-hole for supply formed in the covering member,

the collection portion including a though-hole for collection formed in the covering member so as to be connected to a portion on the most downstream side in a flowing direction of the fluid to-be-treated in the channel,

the plurality of fluids to-be-treated being respectively caused to flow from the plurality of supply portions into the channel, the plurality of fluids to-be-treated caused to flow in being merged and subjected to a predetermined treatment, and the treated fluid is drawn from the collection portion to the outside, and

the supply portion side electrode and the collection portion side electrode being sintered simultaneously with the base, and capillary migration being performed by applying a voltage between the supply portion side electrode and the collection portion side electrode.

According to the invention, the fluids to-be-treated that are supplied from the plurality of supply portions flow through the channel by capillary migration and are drawn from the collection portion to the outside. Therefore, when the plurality of fluids to-be-treated that are different from each other are caused to flow in from the plurality of supply portions, respectively, the plurality of fluids to-be-treated caused to flow in are merged and flow through the channel, and are subjected to a predetermined treatment. Then, the treated fluid is drawn from the collection portion to the outside.

In the invention, the supply portion side electrode and the collection portion side electrode which are used for capillary migration, are sintered simultaneously with the base made of ceramics, and therefore the adhesiveness between the electrodes and the base is improved. Thus, a portion where the base and the electrodes are adhered is prevented from being corroded by the fluid to-be-treated, in particular, chemicals, and thus the chemical resistance can be improved. Thus, a microchemical chip having wide applicability in which there is no limitation regarding the fluid to-be-treated and supplied can be realized.

In the invention, the supply portion side electrode is formed on an inner circumferential surface of the through-hole for supply formed in the covering member, or on a part of a bottom face of the groove portion formed in the base body which part is to be positioned immediately below the through-hole for supply.

In the invention, the collection portion side electrode is formed on an inner circumferential surface of the through-hole for collection formed in the covering member, or on a part of a bottom face of the groove portion formed in the base body which part is to be positioned immediately below the through-hole for collection.

According to the invention, the electrodes are formed on the bottom face of the groove portion which is a flat surface or on the inner circumferential surface of the through-hole, so that the adhesiveness between the base and the electrodes can be further improved. Furthermore, the electrodes can be formed relatively easily.

In the invention, an agitation portion for agitating the fluids to-be-treated is formed on a downstream side in the flowing direction of the fluid to-be-treated with respect to a position where the channel and the supply portions are connected.

According to the invention, after the plurality of fluids to-be-treated are merged into one, a turbulent flow is generated in the merged fluids to-be-treated by the agitation portion. Thereby, the plurality of fluids to-be-treated can be mixed. Thus, the plurality of fluids to-be-treated can be mixed sufficiently in a shorter channel in comparison with the case of mixing them by diffusion only. Accordingly, the length of the channel can be reduced. It is therefore possible to attain the reduction in the size of the microchemical chip, and to attain the reduction in the size of a microchemical system using the microchemical chip. Furthermore, the predetermined treatment is performed in a state where the plurality of fluids to-be-treated are mixed sufficiently. Therefore, the predetermined treatment can be performed more reliably in comparison with the case where the mixing is insufficient.

In the invention, a cross-section area of the channel and the supply channels is 2.5×10⁻mm² or more and 1 mm² or less.

In the invention, a width of the channel and the supply channels is 50 to 1000 μm.

In the invention, the channel and the supply channels have a rectangular cross-sectional shape and a relationship between a longer side as a width and a shorter side as a depth satisfies the following equation:

$$\frac{length of the shorter side}{length of the longer side} \geq 0.4$$

According to the invention, the cross-section area, the width or the like of the channel and the supply channels is set mentioned above, so that specimens, reagents, or cleaning liquids poured from the supply portions can be efficiently delivered and mixed.

In the invention, the base has a treatment portion for performing a predetermined treatment with respect to the merged fluids to-be-treated, the treatment portion being disposed on a downstream side in the flowing direction of the fluids to-be-treated with respect to a position where the supply portion and the channel are connected, and on an upstream side with respect to the collection portion.

According to the invention, the plurality of fluids to-be-treated caused to flow from a plurality of supply portions, respectively, into the channel are merged and subjected to a predetermined treatment in the treatment portion. Therefore, for example, a reaction product can be obtained by providing two supply portions, and casing a compound that is a raw material to flow in from one supply portion, causing a reagent to flow in from the other supply portion, merging the compound and the reagent and heating the same in the treatment portion to cause a reaction.

The invention provides a method for producing the microchemical chip mentioned above, comprising:

forming a groove portion constituting the channel and the supply channel on a surface of a ceramic green sheet constituting the base body;

forming the through-hole for supply and the through-hole for collection in the covering member;

forming the supply portion side electrode on a part of a bottom face of the groove portion formed in the ceramic green sheet which part is to be positioned immediately below the through-hole for supply, and forming the collection portion side electrode on a part of a bottom face of the groove portion which part is to be positioned immediately below the through-hole for collection;

forming the base body by sintering the ceramic green sheet in which the groove portion, the supply portion side and the collection portion side electrodes are formed at a predetermined temperature; and

forming the base by covering the groove portion on the surface of the base body with the covering member.

According to the invention, the groove portion is formed by pressing a pattern on the surface of the ceramic green sheet constituting the base body, the supply portion side electrode is formed on the part of the bottom face of the groove portion which part is to be positioned immediately below the through-hole for supply, and the collection portion side electrode is formed on the part of the bottom face of the groove portion which part is to be positioned immediately below the through-hole for collection. The through-hole for supply and the through-hole for collection are formed in the covering member.

Then, the base body is formed by sintering the ceramic green sheet having the groove portion, the supply portion side electrode and the collection portion side electrode, at the predetermined temperature, and the base is formed by covering the groove portion on the surface of the base body with the covering member.

By forming the base in this manner, a microchemical chip formed by sintering the supply portion side electrode and the collection portion side electrode simultaneously with the base body can be produced.

The invention provides a method for producing the microchemical chip mentioned above, comprising:

forming a groove portion constituting the channel and the supply channel on a surface of a first ceramic green sheet constituting the base body;

forming the through-hole for supply and the through-hole for collection in a second ceramic green sheet constituting the covering member;

forming the supply portion side electrode on a part of a bottom face of the groove portion formed in the first ceramic green sheet which part is to be positioned immediately below the through-hole for supply, or on an inner circumferential surface of the through-hole for supply formed in the second ceramic green sheet;

forming the collection portion side electrode on a part of a bottom face of the groove portion formed in the first ceramic green sheet which part is to be positioned immediately below the through-hole for collection, or on an inner circumferential surface of the through-hole for collection formed in the second ceramic green sheet;

laminating the second ceramic green sheet on the surface of the first ceramic green sheet having the groove portion so as to cover the groove portion; and

forming the base by sintering the laminated ceramic green sheets at a predetermined temperature.

According to the invention, the groove portion is formed by pressing with a pattern on the surface of the first ceramic green sheet constituting the base body, and the through-hole for supply and the through-hole for collection are formed in the second ceramic green sheet constituting the covering member.

Next, the supply portion side electrode is formed on the part of the bottom face of the groove portion formed in the first ceramic green sheet which part is to be positioned immediately below the through-hole for supply, or on the inner circumferential surface of the through-hole for supply formed in the second ceramic green sheet. The collection portion side electrode is formed on the part of the bottom face of the groove portion formed in the first ceramic green sheet which part is to be positioned immediately below the through-hole for collection, or on the inner circumferential surface of the through-hole for collection formed in the second ceramic green sheet.

Then, the second ceramic green sheet is laminated on the surface of the first ceramic green sheet having the groove portion so as to cover the groove portion, and the base is formed by sintering the laminated ceramic green sheets at the predetermined temperature.

By forming the base in this manner, a microchemical chip formed by sintering the supply portion side electrode and the collection portion side electrode simultaneously with the base can be produced.

### BRIEF DESCRIPTION OF THE DRAWINGS

Other and further objects, features, and advantages of the invention will be more explicit from the following detailed description taken with reference to the drawings wherein:

FIG. 1A is a plan view showing a simplified structure of a microchemical chip according to one embodiment of the invention, and FIG. 1B is a cross-sectional view showing cross-sectional structures taken along sectional lines I-I, II-II, and III-III of the microchemical chip indicated in FIG. 1A;

FIG. 2 is an enlarged perspective view showing a supply port and its vicinity;

FIGS. 3A and 3B are plan views showing states of the processed ceramic green sheets;

FIG. 4 is a fragmentary cross-sectional view showing a state where the ceramic green sheets are laminated;

FIG. 5 is a plan view showing a simplified structure of a lid;

FIG. 6A is a plan view showing a simplified structure of a microchemical chip according to another embodiment of the invention, and FIG. 6B is a cross-sectional view showing cross-sectional structures taken along sectional lines IV-IV, V-V, and VI-VI of the microchemical chip indicated in FIG. 1A;

FIG. 7A is a plan view showing a simplified structure of a microchemical chip according to still another embodiment of the invention, and FIG. 7B is a cross-sectional view showing cross-sectional structures taken along sectional lines VII-VII, VIII-VIII, and IX-IX of the microchemical chip indicated in FIG. 7A; and

FIGS. 8A and 8B are partial enlarged perspective views showing formation embodiments of a supply portion side electrode.

### DETAILED DESCRIPTION

Now referring to the drawings, preferred embodiments of the invention are described below.

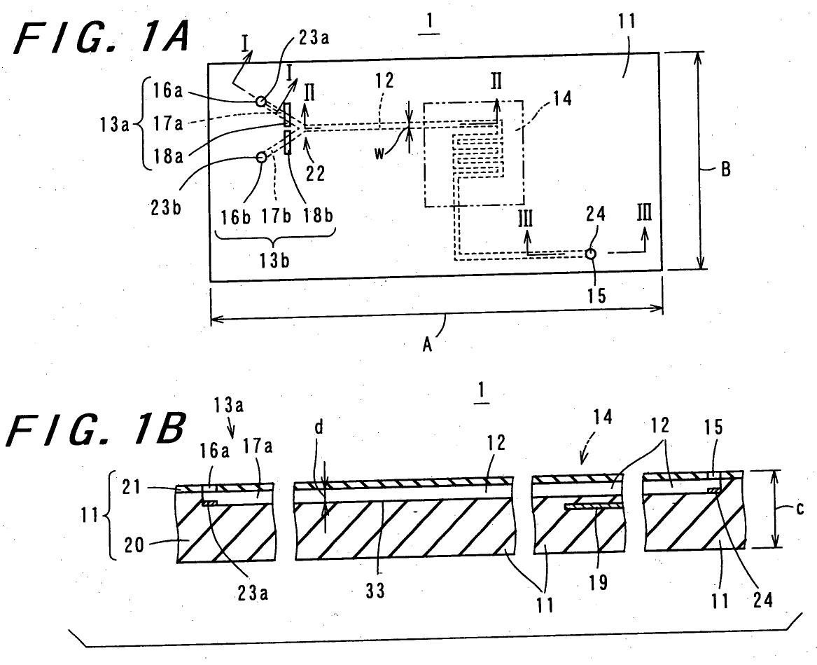

FIG. 1A is a plan view showing a simplified structure of a microchemical chip 1 according to one embodiment of the invention. FIG. 1B is a partial cross-sectional view showing cross-sectional structures taken along sectional lines I-I, II-II, and III-III of the microchemical chip 1 indicated in FIG. 1A. In FIG. 1B, the cross-sectional structures taken along the sectional lines I-I, II-II and III-III are shown in this order.

The microchemical chip 1 comprises a base 11 including a channel 12 for causing a fluid to-be-treated to flow therethrough, two supply portions 13a and 13b each for causing a fluid to-be-treated to flow therefrom into the channel 12, a treatment portion 14 for performing a predetermined treatment to the fluids to-be-treated, and a collection portion 15 from which the treated fluid is drawn to the outside. The base 11 includes a base body 20 made of ceramics, on one surface of which a groove portion 33 is formed, and a lid 21 made of glass that is a covering portion. The channel 12 is formed by covering the surface of the base body 20 having the groove portion 33 with the lid 21.

The supply portion 13a includes a supply channel 17a which is connected to the channel 12, a supply port 16a which is provided at an end portion of the supply channel 17a, and a micropump 18a which is provided on an upstream side in a flowing direction of the fluid to-be-treated with respect to a connecting position 22 to the channel 12. Similarly, the supply portion 13b includes a supply channel 17b, a supply port 16b, and a micropump 18b. The supply ports 16a and 16b are realized as through-holes such that a fluid to-be-treated can be poured into the supply channels 17a and 17b from the outside. The collection portion 15 is realized as a through-hole such that a fluid to-be-treated is removed from the channel 12 to the outside.

A heater 19 is disposed within the base body 20 and at a part of the treatment portion 14 below the channel 12. The channel 12 in the treatment portion 14 is bent and formed in, for example, a zigzag shape so as to pass above heater 19 a plurality of times. A wiring line (not shown) for connecting the heater 19 and an external power source is led out of the heater 19 onto the surface of the base 11. This wiring line is formed of a metal material having a lower electrical resistivity than that of the heater 19.

In the microchemical chip 1, two types of fluids to-be-treated are caused to flow from the two supply portions 13a and 13b into the channel 12, respectively, and are merged into one, and the channel 12 is heated at a predetermined temperature with the heater 19 in the treatment portion 14, if necessary, so that the two types of fluids to-be-treated caused to flow in are reacted, and then the obtained reaction product is drawn from the collection portion 15.

In the microchemical chip 1, supply portion side electrodes 23a and 23b are formed in the supply portions 13a and 13b, and a collection portion side electrode 24 is formed in the collection portion 15. Capillary migration is performed by applying a predetermined voltage between the supply portion side electrodes 23a and 23b and the collection portion side electrode 24.

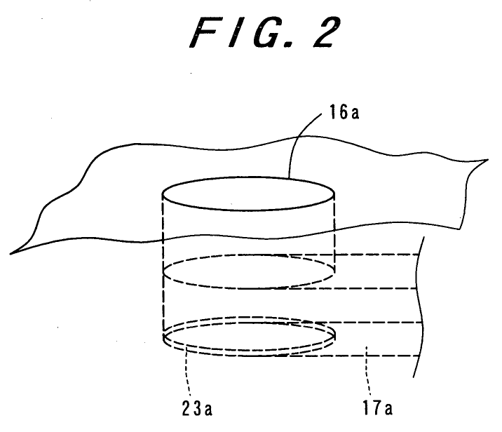

As shown in FIGS. 1A and 1B and FIG. 2, the supply portion side electrode 23a is formed in the supply channel 17a formed in the base 11, more specifically, on a part of a bottom face of a groove portion 33 formed in the base body 20 which part is to be positioned immediately below the supply port 16a, which is a through-hole for supply formed in the lid 21. Similarly to the supply portion side electrode 23a, as shown in FIGS. 1A and 1B, the supply portion side electrode 23b is formed in the supply channel 17b formed in the base 11, more specifically, on a part of a bottom face of the groove portion 33 formed in the base body 20 which part is to be positioned immediately below the supply port 16b, which is a through-hole for supply formed in the lid 21.

As shown in FIGS. 1A and 1B, the collection portion side electrode 24 is formed on a part on the most downstream in the flowing direction of the fluid to-be-treated of a bottom face of the channel 12 formed in the base 11 which part is to be positioned immediately below the through-hole 15, which is a through-hole for collection formed in the lid 21.

The supply portion side electrodes 23a and 23b and the collection portion side electrode 24 are formed by being sintered at the same time when ceramic green sheets are fired and sintered to form the base body 20, as described later. Thus, the adhesiveness between the electrodes 23a, 23b and 24 and the base body 20 is improved. Therefore, a portion where the base body 20 and the electrodes 23a, 23b and 24 are adhered is prevented from being corroded by a fluid to-be-treated, in particular, chemicals, and the chemical resistance can be improved, and thus a microchemical chip 1 having wide applicability in which there is no limitation regarding the fluid to-be-treated and supplied can be realized.

The electrodes 23a, 23b and 24 are formed on the bottom face of the groove portion 33, which is a flat surface, so that the adhesiveness with the base body 20 can be further improved. Moreover, the electrodes 23a, 23b and 24 can be formed relatively easily.

The cross-section area of the channel 12 and the supply channels 17a and 17b is preferably 2.5×10⁻³ mm² or more and 1 mm² or less in order to efficiently deliver and mix specimens, reagents, or cleaning liquids caused to flow in from the supply portions 13a and 13b. However, a fluid flowing through the channel having a whose cross-section area of about 2.5×10⁻³ mm² to 1 mm² generally flows in a laminar flow state, so that simply connecting the two supply channels 17a and 17b allows the two types of fluids to-be-treated that are caused to flow from the supply portions 13a and 13b into the channel 12 and merged to be mixed only by diffusion. Therefore, in order to mix the merged two types of fluids to-be-treated fully, it is necessary to provide a long channel, which limits the achievement of a compact microchemical chip.

In this regards, an agitation portion for agitating the fluids to-be-treated may be formed on the downstream side in the flowing direction of the fluid to-be-treated with respect to the connecting position 22 between the channel 12 and the supply portions 13a and 13b. The agitation portion may be realized by, for example, forming in the channel 12 an uneven portion having an uneven wall surface, a hydrophilic portion having a hydrophilic wall surface or a hydrophobic portion having a hydrophobic wall surface, by arranging a vibration element for imparting vibrations to the fluids to-be-treated in the channel 12, or by bending the channel 12. Thus, after the plurality of fluids to-be-treated are merged into one, a turbulent flow is generated in the merged fluids to-be-treated by the agitation portion.

In this manner, the plurality of fluids to-be-treated can be mixed by generating the turbulent flow in the merged fluids to-be-treated. Thus, the plurality of fluids to-be-treated can be mixed sufficiently in a shorter channel in comparison with the case of mixing them by diffusion only. Accordingly, the length of the channel 12 can be reduced. It is therefore possible to attain the reduction in the size of the microchemical chip 1, and to attain the reduction in the size of a microchemical system using the microchemical chip 1. Furthermore, the predetermined treatment is performed in a state where the plurality of fluids to-be-treated are mixed sufficiently. Therefore, the predetermined treatment can be performed more reliably in comparison with the case where the mixing is insufficient.

Furthermore, by forming the agitation portion between the connecting position 22 and the treatment portion 14, the merged fluids to-be-treated have been mixed sufficiently in arriving at the the treatment portion 14. Therefore, for example, in a case where a compound serving as a raw material is caused to flow in from the supply portion 13a, where a reagent is caused to flow in from the supply portion 13b, and where the compound and the reagent are merged and are reacted by heating them with the heater 19 of the treatment portion 14, the compound and the reagent can be heated in a state where the compound and the reagent are mixed sufficiently. It is therefore possible to efficiently react the compound and the reagent, and to enhance the the yield of a reaction product which is taken out of the collection portion.

Since the base body 20 is made of a ceramic material, the base body has excellent chemical resistance, compared with silicon, glass or resin, so that a microchemical chip 1 that can be used under various conditions can be obtained. Examples of the ceramic material constituting the base body 20 include an aluminum oxide sintered substance, a mullite sintered substance or a glass ceramic sintered substance. The lid 21 is made of glass and therefore the mixture state or the reaction state of the fluid to-be-treated can be visually confirmed.

The cross-section area of the channel 12 and the supply channels 17a and 17b is preferably 2.5×10⁻³ mm² or more and 1 mm² or less in order to efficiently deliver and mix specimens, reagents, or cleaning liquids caused to flow in from the supply portions 13a and 13b mentioned above. When the cross-section area of the channel 12 and the supply channels 17a and 17b exceeds 1 mm², the amount of delivered specimen, reagent, or cleaning liquid becomes excessive, so that an effect of increasing a reaction surface area per unit volume and reducing the reaction time significantly of the microchemical chip 1 cannot sufficiently be obtained. Furthermore, when the cross-section area of the channel 12 and the supply channels 17a and 17b is less than 2.5×10⁻³ mm², the loss of the pressure due to the micropumps 18a and 18b is increased, so that a problem is caused in delivering fluids. Therefore, it is preferable that the cross-section area of the channel 12 and the supply channels 17a and 17b is 2.5×10⁻³ mm² or more and 1 mm² or less.

The width w of the channel 12 and the supply channels 17a and 17b is preferably 50 to 1000 μm, more preferably 100 to 500 μm. The depth d of the channel 12 and the supply channels 17a and 17b is preferably 50 to 1000 μm, more preferably 100 to 500 μm, and within the preferable range of the cross-section area as described above. When the channel 12 and the supply channels 17a and 17b have a rectangular cross-sectional shape, the relationship between the width (a longer side) and the depth (a shorter side) preferably satisfies the following equation:

$$\frac{length of the shorter side}{length of the longer side} \geq 0.4 , \\ more preferably , \\ \frac{length of the shorter side}{length of the longer side} \geq 0.6 .$$

In the case of a ratio of the length of the shorter side to the length of the longer side <0.4, the pressure loss is large, which causes a problem in delivering fluids.

The outline size of the microchemical chip 1 is, for example, such that the width A is about 40 mm, the depth B is about 70 mm, and the height C is about 1 to 2 mm, but the invention is not limited thereto, and an appropriate outline size can be used, depending on the necessity.

The microchemical chip 1 after use can be reused, when the microchemical chip 1 is cleaned by poring a cleaning liquid from the supply portions 13a and 13b.

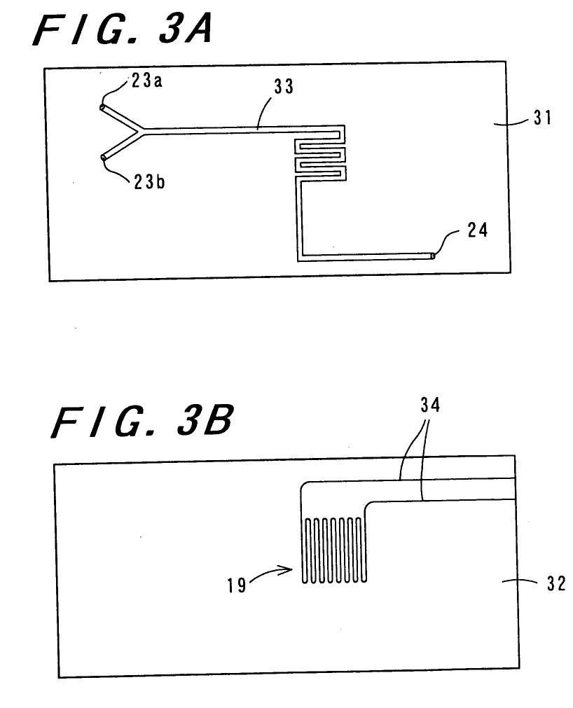

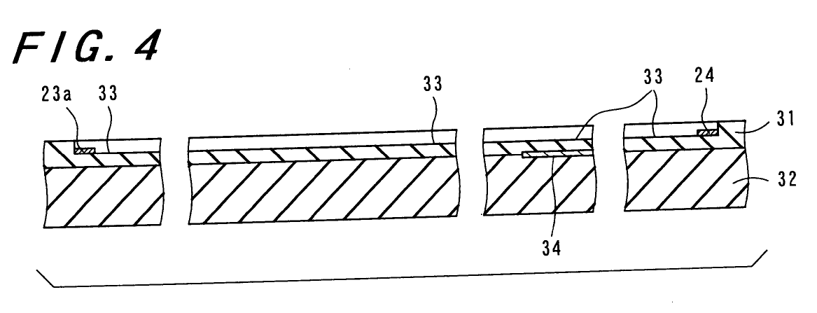

Next, a method for producing the microchemical chip 1 shown in FIGS. 1A and 1B will be described. FIGS. 3A and 3B are plan views showing states of the processed ceramic green sheets 31 and 32. FIG. 4 is a cross-sectional view showing a state where the ceramic green sheets 31 and 32 are laminated.

First, a suitable organic binder and solvent are mixed with a raw material powder, and if necessary, a plasticizer or a dispersant is added thereto, and the mixture is formed into a slurry. Then, the slurry is molded into a sheet by doctor blading, calendar rolling or the like. Thus, a ceramic green sheet (also referred to as “ceramic crude sheet”) is formed. As the raw material powder, for example, when the base body 20 is made of an aluminum oxide sintered substance, aluminum oxide, silicon oxide, magnesium oxide, calcium oxide or the like can be used.

In this embodiment, two of the thus formed ceramic green sheets are used to form the base body 20. First, as shown in FIG. 3A, a groove portion 33 is formed by pressing with a pattern on a surface of the first ceramic green sheet 31 with a pattern. In this case, a pattern having a shape to which a desired shape of the groove portion 33 is transferred is used. Incidentally, by using a pattern in which an uneven shape is transferred on a portion corresponding to a predetermined wall surface part, as the shape of the groove portion, unevenness can be formed on a wall surface part of the groove portion which constitutes the uneven portion serving as the agitation portion stated before.

The pressing pressure for pressing the slurry with the pattern is adjusted depending on the viscosity of the slurry before being molded into the ceramic green sheet. For example, when the viscosity of the slurry is 1 to 4 Pa.s, a pressure of 2.5 to 7 MPa is applied to the slurry. There is no particular limitation regarding the material of the pattern, and a metal pattern or a wooden pattern can be used.

Furthermore, the supply portion side electrodes 23a and 23b and the collection portion side electrode 24 are formed on the first ceramic green sheet 31 in which the groove portion 33 is formed by processing for forming a thin film. The supply portion side electrode 23a is formed on the bottom face of a part corresponding to the supply channel 17a in the groove portion 33 which part is a part on the most upstream side in the flowing direction of the fluid to-be-treated, that is, a part of the bottom face which part is to be positioned immediately below the supply port 16a, which is a through-hole for supply formed in the lid 21. The supply portion side electrode 23b is formed on the bottom face of a part corresponding to the supply channel 17b in the groove portion 33 which part is a part on the most upstream side in the flowing direction of the fluid to-be-treated, that is, a part of the bottom face which part is to be positioned immediately below the supply port 16b, which is a through-hole for supply formed in the lid 21. The collection portion side electrode 24 is formed on a part of the bottom face on the most downstream side in the flowing direction of the fluid to-be-treated in the groove portion 33 which part is to be positioned immediately below the collection portion 15, which is a through-hole for collection formed in the lid 21. Furthermore, a wiring pattern (not shown) that is connected to each of the electrodes 23a, 23b and 24 is formed on the surface of the first ceramic green sheet 31 by processing for forming a thin film, which is the same manner as for the electrodes.

As shown in FIG. 3B, a wiring pattern 34 constituting the heater 19 and a wiring line for external power connection is formed on the surface of the second ceramic green sheet 32 by applying a conductive paste in a predetermined shape by screen printing or the like. The conductive paste can be obtained by mixing a metal material powder such as tungsten, molybdenum, manganese, copper, silver, nickel, palladium, or gold with a suitable organic binder and solvent. For the conductive paste for forming the wiring pattern 34 constituting the heater 19, a conductive paste in which 5 to 30 wt % of a ceramic powder is added to a metal material powder as described above such that a predetermined electric resistance value is achieved after firing is used.

As shown in FIG. 4, the first ceramic green sheet 31 having the groove portion 33 and the electrodes 23a, 23b and 24 is laminated on the surface of the second ceramic green sheet 32 having the wiring pattern 34 constituting the heater 19. The laminated first and second ceramic green sheets 31 and 32 are sintered at a temperature of about 1600° C. Thus, the base body 20 shown in FIGS. 1A and 1B in which the electrodes 23a, 23b and 24 are formed on the bottom face of the groove portion 33 is formed.

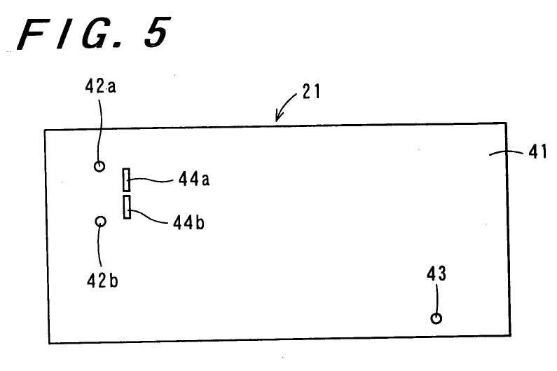

FIG. 5 is a plan view showing a simplified structure of the lid 21. As shown in FIG. 5, through-holes 42a, 42b and 43 that are in communication with the groove portion 33 of the first ceramic green sheet 31 shown in FIG. 3A are formed in the predetermined positions constituting the supply ports 16a and 16b and the collection portion 15 in a substrate 41 made of glass, and thus the lid 21 can be obtained.

The lid 21 is bonded onto the surface on which the groove portion 33 is exposed of the base body 20. The lid 21 and the base body 20 are bonded by heating and pressing.

Next, piezoelectric materials 44a and 44b such as lead zirconate titanate (PZT; composition formula: Pb(Zr, Ti)O₃) are attached into predetermined positions on the surface of the lid 21, and wiring lines (not shown) for applying a voltage to the piezoelectric materials 44a and 44b are formed. The piezoelectric materials 44a and 44b can vibrate the lid 21 above the supply channels 17a and 17b by expanding or contracting in accordance with the applied voltage, and therefore micropumps 18a and 18b for delivering fluids can be formed by attaching the piezoelectric materials 44a and 44b to the lid 21 above the supply channels 17a and 17b.

In the manner described above, the base 11 shown in FIGS. 1A and 1B is formed so that the microchemical chip 1 can be obtained. Thus, the microchemical chip 1 including the supply portion side electrodes 23a, and 23b and the collection portion side electrode 24 that are used for capillary migration can be produced by attaching the base body 20 in which the electrodes 23a, 23b and 24 are formed on the bottom face of the groove portion 33, and the lid 21.

In this embodiment, the base body 20 is formed by sintering a laminated structure which consists of the ceramic green sheet 31 on the surface of which the groove portion 33 is formed by pressing with a pattern and the ceramic green sheet 32 having the wiring pattern 34 constituting the heater 19, whereupon the base 11 having the channel 12 is formed by covering the groove portion 33 on the surface of the base body 20 with the lid 21. Therefore, the microchemical chip 1 can be produced only by simple processing without performing complicated processing such as etching processing that is necessary when forming a channel in a base 11 made of silicon, glass or resin.

As described above, although the microchemical chip 1 of this embodiment has two supply portions 13a and 13b, the invention is not limited thereto, and the microchemical chip 1 can have three or more supply portions. When two or more supply portions are provided, the supply portions are not necessarily provided so as to be merged in one portion, but can be connected to the channel 12 at different positions. In this case, an electrode used for capillary migration is formed in each supply portion.

This embodiment has a structure in which one treatment portion 14 (heater 19) is provided, but the invention is not limited thereto and two or more treatment portions (heaters) are provided. Thus, a complicated reaction can be controlled by providing three or more supply portions and two or more treatment portions (heaters). It should be noted that it is not necessary to provide the treatment portion 14 (heater 19) when a reaction can proceed without heating.

In the microchemical chip 1 of this embodiment, the collection portion 15 is provided and a reaction product is drawn from the collection portion 15. When a detecting portion is provided in the collection portion 15 or on the upstream side in the flowing direction of the fluid to-be-treated with respect to the collection portion 15, a reaction product of a chemical reaction or a biochemical reaction such as an antigen-antibody reaction and an enzyme reaction can be detected.

Furthermore, this embodiment is configured to have the micropumps 18a and 18b as means for delivering fluids, but it is possible not to provide the micropumps 18a and 18b. In this case, when pouring a fluid to-be-treated from the supply ports 16a and 16b, the fluid can be delivered from the supply ports 16a and 16b to the collection portion 15 by forcing the fluid in with a microsyringe or the like. Alternatively, when pouring a fluid, the fluid can be delivered by pouring the fluid under application of pressure with a pump or the like provided outside. In addition, the fluid to-be-treated can be delivered by suction with a microsyringe or the like from the collection portion 15 that is realized as an opening, after the fluid to-be-treated is poured from the supply ports 16a and 16b.

The lid 21 is bonded to the base body 20, but the invention is not limited thereto, and the lid 21 can be provided removably from the base body 20. For example, a structure where pressure is applied to the entire microchemical chip with a silicone rubber sandwiched by the base body 20 and the lid 21 is possible.

In the method for producing the microchemical chip 1 of this embodiment, the base body 20 is formed with two ceramic green sheets, that is, the ceramic green sheet 31 having the groove portion 33 and the ceramic green sheet 32 having the wiring pattern 34 constituting the heater 19. However, the invention is not limited thereto, and the base body 20 can be formed with three or more ceramic green sheets.

In this embodiment, the base 11 is formed by sintering with the groove portion 33 on the surface of the ceramic green sheet 31 exposed to form the base body 20 and then covering the groove portion 33 on the surface of the base body 20 with the lid 21. However, the invention is not limited thereto. The base 11 can be formed by further laminating a ceramic green sheet provided with the same through-hole as in the lid 21 that is in communication with the groove portion 33 on the surface of the ceramic green sheet 31 and sintering the ceramic green sheets.

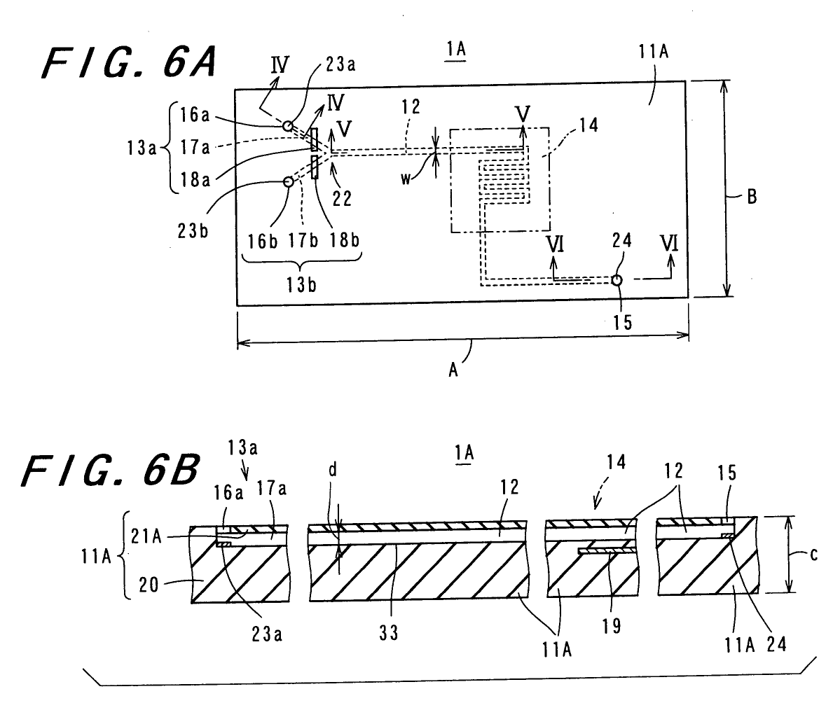

FIG. 6A is a plan view showing a simplified structure of a microchemical chip 1A according to another embodiment of the invention. FIG. 6B is a cross-sectional view showing cross-sectional structures taken along sectional lines IV-IV, V-V, and VI-VI of the microchemical chip 1A indicated in FIG. 6A. In FIG. 6B, the cross-sectional structures taken along the sectional lines IV-IV, V-V and VI-VI are shown in this order. Furthermore, in this embodiment, the same components as those of the aforementioned embodiment will be denoted by the same reference numerals, and it will be omitted to describe in detail.

The base 11A of the microchemical chip 1A of the embodiment includes a base body 20 made of ceramics and a lid 21A made of glass that is a covering portion, and the channel 12 is formed by covering the surface of the base body 20 which surface has the groove portion 33 with the lid 21A. Here, the base body 20 and the lid 21A are formed integrally. In the lid 21A, like the lid 21 of the embodiment mentioned above, the supply ports 16a and 16b which are through-holes for supply and the collection portion 15 realized as a through-hole, which is a through-hole for collection, are formed.

Such a base 11A is formed described below. Similarly to the embodiment mentioned above, as shown in FIG. 3A, the groove portion 33 constituting the channel 12 and the supply channels 17a and 17b is formed on the surface of the ceramic green sheet 31. Moreover, Similarly to the embodiment mentioned above, the supply portion side electrode 23a and 23b and the collection side electrode 24 are formed on the bottom face of the groove portion 33. Next, as shown in FIG. 3B, the wiring pattern 34 constituting the heater 19 and the wiring line for external power connection is formed on the surface of the ceramic green sheet 32.

Next, Similarly to the lid 21 shown in FIG. 5, the through-holes 42a and 42b which are through-holes for supply and the through-hole 43 which is a through-hole for collection, are formed in the ceramic green sheet constituting the lid 21A.

Next, as shown in FIG. 4, the ceramic green sheet 31 having the groove portion 33 and the electrodes 23a, 23b and 24 is laminated on the surface of the ceramic green sheet 32 having the wiring pattern 34 constituting the heater 19. Moreover, the ceramic green sheet having the through-holes 42a and 42b and 43, is laminated on the surface of the ceramic green sheet 31 having the groove portion 33 and the electrodes 23a, 23b and 24, so as to cover the groove portion 33. The laminated three ceramic green sheets including the ceramic green sheets 31 and 32 and the other ceramic green sheet are sintered at a temperature of about 1600° C. Thus, the base 11A shown in FIGS. 6A and 6B in which the electrodes 23a, 23b and 24 are formed on the bottom face of the groove portion 33, is formed.

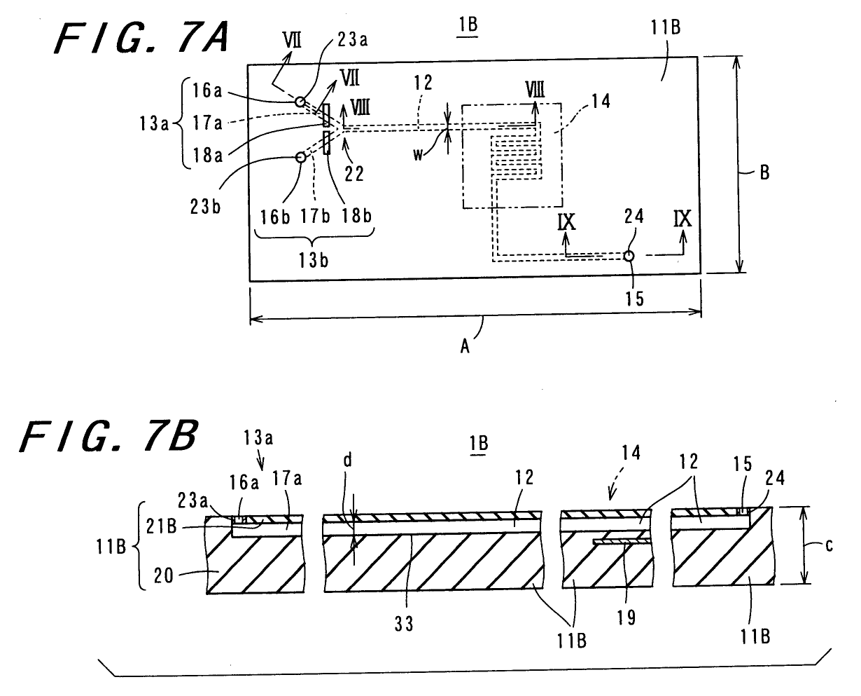

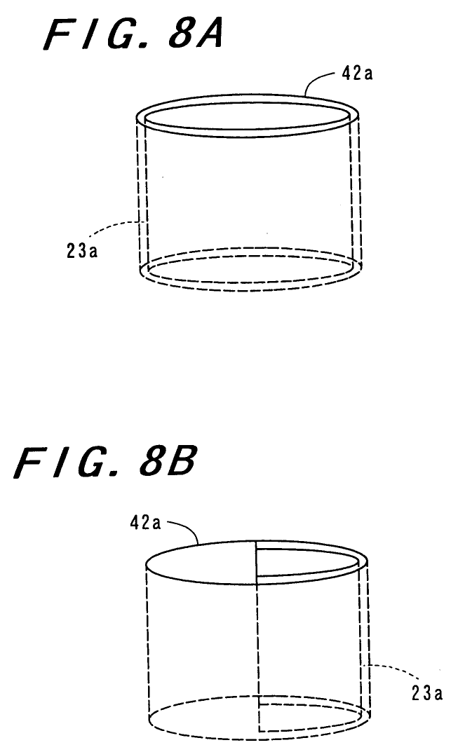

FIG. 7A is a plan view showing a simplified structure of a microchemical chip according to still another embodiment of the invention. FIG. 7B is a cross-sectional view showing cross-sectional structures taken along sectional lines VII-VII, VIII-VIII, and IX-IX of the microchemical chip indicated in FIG. 7A. In FIG. 7B, the cross-sectional structures taken along the sectional lines VII-VII, VIII-VIII, and IX-IX are shown in this order. FIGS. 8A and 8B are partial enlarged perspective views showing formation embodiments of the supply portion side electrodes 23a. Furthermore, in this embodiment, the same components as those of the aforementioned embodiment will be denoted by the same reference numerals, and it will be omitted to describe in detail.

In case where the lid is formed of a ceramic green sheet, the electrodes 23a, 23b and 24 may be formed in the lid 21B, instead of the base body 20. That is, as shown in FIGS. 7A, 7B and 8A, the supply portion side electrodes 23a and 23b are formed on the whole inner circumferential surface of the through-holes for supply 42a and 42b formed in the lid 21B of the base 11B, and the collection portion side electrode 24 is formed on the whole inner circumferential surface of the through-hole for collection 43 formed in the lid 21B of the base 11B.

Such a base 11B is formed described below. Similarly to the embodiment mentioned above, the groove portion 33 constituting the channel 12 and the supply channels 17a and 17b is formed on the surface of the ceramic green sheet 31. Next, as shown in FIG. 3B, the wiring pattern 34 constituting the heater 19 and the wiring line for external power connection is formed on the surface of the ceramic green sheet 32.

Next, Similarly to the lid 21 shown in FIG. 5, the through-holes 42a and 42b which are through-holes for supply and the through-hole 43 which is a through-hole for collection, are formed in the ceramic green sheet constituting the lid 21B. The supply portion side electrodes 23a and 23b are formed on the whole inner circumferential surface of the through-holes 42a and 42b, and the collection portion side electrode 24 is formed on the whole inner circumferential surface of the through-hole 43.

Next, the ceramic green sheet 31 having the groove portion 33 is laminated on the surface of the ceramic green sheet 32 having the wiring pattern constituting the heater 19. Moreover, the ceramic green sheet having the through-holes 42a, 42b and 43 is laminated on the surface of the ceramic green sheet 31 having the groove portion 33 so as to cover the groove portion 33. The laminated three ceramic green sheets 31 and 32 are sintered at a temperature of about 1600° C. Thus, the base 11B shown in FIGS. 7A, 7B and 8A in which the electrodes 23a, 23b and 24 are formed on the whole inner circumferential surfaces of the through-holes 42a, 42b and 43, is formed.

Note that, in this embodiment, the electrodes 23a, 23b and 24 are formed on the whole inner circumferential surface of the through-holes 42a, 42b and 43, however, instead of those, as shown in FIG. 8B, the electrodes 23a, 23b and 24 may be formed on the halves of the inner circumferential surfaces of the through-holes 42a, 42b and 43, respectively.

When the bases 11A and 11B are formed by laminating the ceramic green sheets in this manner, it is not necessary to attach the lid 21 after the base body 20 is formed, so that the productivity can be improved. In the case where a ceramic piezoelectric material such as PZT as described above is used for the piezoelectric materials 44a and 44b constituting the micropumps 18a and 18b, after the ceramic piezoelectric material is attached in a predetermined position in the ceramic green sheet in which the through-hole in communication with the groove portion 33 is formed, the piezoelectric material can be sintered at the same time.

The microchemical chip of the invention can be used for applications such as tests of viruses, bacteria or humor components in humors such as blood, saliva and urine with a reagent, vital reaction experiments between viruses, bacteria or medical fluid and body cells, reaction experiments between viruses or bacteria and medical fluid, reaction experiments between viruses or bacteria and other viruses or bacteria, blood identification, separation and extraction or decomposition of genes with medical fluid, separation and extraction by precipitation or the like of a chemical substance in a solution, decomposition of a chemical substance in a solution, and mixture of a plurality of medical fluids, and can be used for the purpose of other vital reactions or chemical reactions.

The invention may be embodied in other specific forms without departing from the spirit or essential characteristics thereof. The present embodiments are therefore to be considered in all respects as illustrative and not restrictive, the scope of the invention being indicated by the appended claims rather than by the foregoing description and all changes which come within the meaning and the range of equivalency of the claims are therefore intended to be embraced therein.

What is claimed is:

1. A microchemical chip comprising:
a base including a channel for causing a fluid to-be-treated to flow therethrough, a plurality of supply portions connected to the channel, for causing a plurality of fluids to-be-treated to flow therefrom into the channel, respectively, and a collection portion which is connected to the channel and from which a fluid in the channel is drawn to the outside;
a supply portion side electrode formed in the supply portion; and
a collection portion side electrode formed in the collection portion,
the base being composed of a base body made of ceramics having a groove portion constituting the channel and a covering member arranged on the base body so as to cover the groove portion,
the supply portion including a supply channel having its one end connected to the channel and its another end connected to a through-hole for supply formed in the covering member,
the collection portion including a though-hole for collection formed in the covering member so as to be connected to a portion on the most downstream side in a flowing direction of the fluid to-be-treated in the channel,
the plurality of fluids to-be-treated being respectively caused to flow from the plurality of supply portions into the channel, the plurality of fluids to-be-treated caused to flow in being merged and subjected to a predetermined treatment, and the treated fluid is drawn from the collection portion to the outside, and
the supply portion side electrode and the collection portion side electrode being sintered simultaneously with the base body, and capillary migration being performed by applying a voltage between the supply portion side electrode and the collection portion side electrode.

2. The microchemical chip of claim 1, wherein an agitation portion for agitating the fluids to-be-treated is formed on a downstream side in the flowing direction of the fluid to-be-treated with respect to a position where the channel and the supply portions are connected.

3. The microchemical chip of claim 1, wherein a cross-section area of the channel and the supply channels is 2.5×10⁻³ mm² or more and 1 mm² or less.

4. The microchemical chip of claim 1, wherein a width of the channel and the supply channels is 50 to 1000 μm.

5. The microchemical chip of claim 1, wherein the channel and the supply channels have a rectangular cross-sectional shape and a relationship between a longer side as a width and a shorter side as a depth satisfies the following equation:
$$\frac{length of the shorter side}{length of the longer side} \geq 0.4$$

6. The microchemical chip of claim 1, wherein the supply portion side electrode is formed on a part of a bottom face of the groove portion formed in the base body which part is to be positioned immediately below the through-hole for supply, and
the collection portion side electrode is formed on a part of a bottom face of the groove portion formed in the base body which part is to be positioned immediately below the through-hole for collection.

7. A microchemical chip comprising:
a base made of ceramics and including a channel for causing a fluid to-be-treated to flow therethrough, a plurality of supply portions connected to the channel, for causing a plurality of fluids to-be-treated to flow therefrom into the channel, respectively, and a collection portion which is connected to the channel and from which a fluid in the channel is drawn to the outside;
a supply portion side electrode formed in the supply portion; and
a collection portion side electrode formed in the collection portion,
the base being composed of a base body made of ceramics having a groove portion constituting the channel and a covering member made of ceramics and arranged on the base body so as to cover the groove portion,
the supply portion including a supply channel having its one end connected to the channel and its another end connected to a through-hole for supply formed in the covering member,
the collection portion including a though-hole for collection formed in the covering member so as to be connected to a portion on the most downstream side in a flowing direction of the fluid to-be-treated in the channel,
the plurality of fluids to-be-treated being respectively caused to flow from the plurality of supply portions into the channel, the plurality of fluids to-be-treated caused to flow in being merged and subjected to a predetermined treatment, and the treated fluid is drawn from the collection portion to the outside, and
the supply portion side electrode and the collection portion side electrode being sintered simultaneously with the base, and capillary migration being performed by applying a voltage between the supply portion side electrode and the collection portion side electrode.

8. The microchemical chip of claim 7, wherein an agitation portion for agitating the fluids to-be-treated is formed on a downstream side in the flowing direction of the fluid to-be-treated with respect to a position where the channel and the supply portions are connected.

9. The microchemical chip of claim 7, wherein a cross-section area of the channel and the supply channels is 2.5×10⁻³ mm² or more and 1 mm² or less.

10. The microchemical chip of claim 7, wherein a width of the channel and the supply channels is 50 to 1000 μm.

11. The microchemical chip of claim 7, wherein the channel and the supply channels have a rectangular cross-sectional shape and a relationship between a longer side as a width and a shorter side as a depth satisfies the following equation:
$$\frac{length of the shorter side}{length of the longer side} \geq 0.4$$

12. The microchemical chip of claim 7, wherein the collection portion side electrode is formed on an inner circumferential surface of the through-hole for collection formed in the covering member, or on a part of a bottom face of the groove portion formed in the base body which part is to be positioned immediately below the through-hole for collection.

13. The microchemical chip of claim 7, wherein the supply portion side electrode is formed on an inner circumferential surface of the through-hole for supply formed in the covering member, or on a part of a bottom face of the groove portion formed in the base body which part is to be positioned immediately below the through-hole for supply.

14. The microchemical chip of claim 13, wherein the collection portion side electrode is formed on an inner circumferential surface of the through-hole for collection formed in the covering member, or on a part of a bottom face of the groove portion formed in the base body which part is to be positioned immediately below the through-hole for collection.

15. The microchemical chip of claim 1, wherein the base has a treatment portion for performing a predetermined treatment with respect to the merged fluids to-be-treated, the treatment portion being disposed on a downstream side in the flowing direction of the fluids to-be-treated with respect to a position where the supply portion and the channel are connected, and on an upstream side with respect to the collection portion.

16. The microchemical chip of claim 7, wherein the base has a treatment portion for performing a predetermined treatment with respect to the merged fluids to-be-treated, the treatment portion being disposed on a downstream side in the flowing direction of the fluids to-be-treated with respect to a position where the supply portion and the channel are connected, and on an upstream side with respect to the collection portion.

17. A method for producing the microchemical chip of claim 1, comprising:
forming a groove portion constituting the channel and the supply channel on a surface of a ceramic green sheet constituting the base body;
forming the through-hole for supply and the through-hole for collection in the covering member;
forming the supply portion side electrode on a part of a bottom face of the groove portion formed in the ceramic green sheet which part is to be positioned immediately below the through-hole for supply, and forming the collection portion side electrode on a part of a bottom face of the groove portion which part is to be positioned immediately below the through-hole for collection;
forming the base body by sintering the ceramic green sheet having the groove portion, the supply portion side and the collection portion side electrodes, at a predetermined temperature; and
forming the base by covering the groove portion on the surface of the base body with the covering member.

18. A method for producing the microchemical chip of claim 7, comprising:
forming a groove portion constituting the channel and the supply channel on a surface of a first ceramic green sheet constituting the base body;
forming the through-hole for supply and the through-hole for collection in a second ceramic green sheet constituting the covering member;
forming the supply portion side electrode on a part of a bottom face of the groove portion formed in the first ceramic green sheet which part is to be positioned immediately below the through-hole for supply, or on an inner circumferential surface of the through-hole for supply formed in the second ceramic green sheet;
forming the collection portion side electrode on a part of a bottom face of the groove portion formed in the first ceramic green sheet which part is to be positioned immediately below the through-hole for collection, or on an inner circumferential surface of the through-hole for collection formed in the second ceramic green sheet;
laminating the second ceramic green sheet on the surface of the first ceramic green sheet having the groove portion so as to cover the groove portion; and
forming the base by sintering the laminated ceramic green sheets at a predetermined temperature.
